(19) United States
(12) Patent Application Publication
(10) Pub. No.: US 2005/0129774 A1
(43) Pub. Date: Jun. 16, 2005

(54) Non-donating nsaids adsorbed into carrier particles

(75) Inventors: Sven Morein, Sodertalje (SE); Mats Berg, Sodertalje (SE); Christina Holmberg, Sodertalje (SE); Per Johan Lundberg, Molndal (SE); Anders Ringberg, Sodertalje (SE)

Correspondence Address: ARENT FOX KINTNER PLOTKIN & KAHN, 1050 CONNECTICUT AVENUE, N.W., SUITE 400, WASHINGTON, DC, 20036, US

(73) Assignees: ASTRAZENECA AB, Sudertalke (SE); ASTRAZENECA UK LIMITED, London (GB)

(21) Appl. No.: 10/507,368

(22) PCT Filed: Mar. 20, 2003
(86) PCT No.: PCT/SE03/00468

(30) Foreign Application Priority Data
Mar. 22, 2002 (SE) 0200895-1

(51) Int. Cl. A61K 9/14

(52) U.S. Cl. 424/489

(57) ABSTRACT

The present invention relates to porous particles comprising one or more NO-donating Non Steroidal Antiinflammatory Compound(s) optionally mixed with one or more surfactant(s) and to new solid drug delivery composition comprising said particles optionally in combination with a second active drug. Futhermore, the invention relates to processes for producing said porous particles and solid drug delivery composition as well as the use of said composition in the manufacturing of a medicament. The No-donating NSAID may be in oily or melted form.

### FIELD OF INVENTION

The present invention relates to porous particles comprising one or more NO-donating Non Steroidal Antiinflammatory Compound(s) (NSAID(s)), optionally mixed with one or more surfactant(s) and to a new solid drug delivery composition comprising said particles optionally in combination with a second active drug.

Futhermore, the invention relates to processes for producing said porous particles and solid drug delivery composition as well as the use of said composition in the manufacturing of a medicament.

### BACKGROUND OF THE INVENTION

Non-steroidal anti-inflammatory drugs, commonly and hereafter abbreviated as NSAIDs, are well-known drugs for the treatment of pain and inflammation. One of the major drawbacks with NSAIDs is that they have severe gastrointestinal side-effects. Patients undergoing treatment with NSAIDs for a longer period of time, such as naproxen, often experience problems with stomach gastrointestinal side-effects.

Nitrogen oxide donating NSAID drugs (in the following NO-donating NSAIDs), have been found to have an improved side-effect profile, see e.g. WO 94/04484, WO 94/12463, WO 95/09831 and WO 95/30641.

NO-donating NSAIDs are lipophilic drugs with poor aqueous solubility. A biopharmaceutical problem with these drugs is that their absorption from the gastrointestinal tract (GIT) may be dissolution rate limited, resulting in poor bioavailibility upon oral administration.

Many of the NO-donating NSAIDs are obtained as such, in the form of an oily compound. Therefore, the conventional methods for formulating these compounds such as tabletting are not applicable for these compounds. Oily active drugs have generally been produced and put on the market in soft gelatine capsules. NO-donating NSAIDs in oily form cannot, in its pure form, be compressed into a conventional tablet.

One advantageous solution to the problem in handling oily substances and to obtain a dosage form for oral administration is forming a Self Emulsifying Drug Delivery System, commonly known as SEDDS, e.g. as described in WO 01/66087. More particularly, the SEDDS is a pharmaceutical composition suitable for oral administration, in the form of an emulsion pre-concentrate, comprising one or more NO-donating NSAID(s); one or more surfactants; and optionally an oil or semi-solid fat. The composition forms in-situ an oil-in-water emulsion upon contact with aqueous media such as gastrointestinal fluids. The pre-concentrate emulsion is usually filled into conventional capsules.

The self-emulsifying process of a SEDDS formulation depends mainly on the characteristics of the oil(s)/surfactant(s) mixture and the amount of surfactant. The polarity of the oil affects the solubility properties and the self-emulsification ability of the oil. In the literature it is suggested to use a surfactant that achieves a hydrophilicity of the emulsion that is thought to be necessary for immediate/rapid formation of an oil-in-water emulsion. SEDDS formulations of NO-donating NSAID(s), containing a surfactant(s) form an emulsion. This depends on the properties of the NO-donating NSAID combined with the properties of the surfactant(s). Aulton, M. E. Pharmaceutics, The science of dosage form design, p. 291, (1988), Gershanik, T.; Benita, S., Eur. J. Pharm. Biopharm., 50, 179 (2000), Bachynsky, M. O.; Shah N. H.; Patel, C. I.; Malick, A. W., Compound. Dev. Ind. Pharm., 23(8), 809 (1997), Pouton, C. W., Adv. Compound Deliv.

Rev., 25, 47 (1997), Shah N. H.; Carvajal, M. T.; Patel, C. I.; Infeld, M. H.; Malick, A. W., Int. J. Pharm., 106, 15 (1994), Costantinides, P. P., Pharm. Res., 12(11), 1561 (1995).

Emulsion or preconcentrates are not the preferred formulations in pharmaceutical industry. One drawback may for example be the stability of such formulations. Tablets and capsules are often preferred in view of large scale manufacturing of drug delivery compositions.

Tabletted compositions comprising an oily, sticky active agent and a method for producing such compositions are described in WO 99/27912 and WO 99/27913. These documents describe absorption of the oily sticky component into a porous carrier. However, compositions comprising NO-donating NSAIDs are not mentioned or proposed in any of these documents and no compressed tablets comprising NO-donating NSAIDs are hitherto known.

One of the unique features of NO-donating NSAIDs is that many of these drugs are oils or thermosoftening semisolids, which are practically insoluble in water. With high-dose NO-donating NSAIDs, e.g. when the dose is above about 350 mg, it is difficult to formulate a tablet of reasonable size of the large amount of oil or semisolid.

In the attempts to make conventional tablets comprising NO-donating NSAIDs, such as NO-donating naproxen, which is a so-called high dose drug, the result has been a too large tablet. The patient compliance will be influenced by a tablet of unacceptable size.

The object of the present invention is to provide solid drug delivery compositions comprising NO-donating NSAID(s) such as tablets and capsules of acceptable size to the patients.

### DETAILED DESCRIPTION OF THE INVENTION

NO-donating NSAIDs are lipophilic drugs with poor aqueous solubility. They can be classified into class 2 according to the Biopharmaceutical Classification System proposed by Amidon et al. (Pharm. Res. 12 (1995) pp. 413-420). Compounds of this class are characterised by their low aqueous solubility but reasonably well permeability. A biopharmaceutical problem with these drugs is that their absorption from the gastrointestinal tract (GIT) may be dissolution rate limited, resulting in poor bioavailibility upon oral administration. One object of the invention is to provide an oral formulation with satisfactory bioavailability.

Active Drug

The wording “NSAID” is defined as a non-steroidal anti-inflammatory drug, i.e. any drug having an anti-inflammatory effect, but which drug does not belong to the drug class “steroids”. A person skilled in the art will recognise a drug that falls under the definition NSAID. Examples of specific NSAIDs are naproxen, diclofenac, aceclofenac, indomethacine, ketorolac, sulindac, meloxicam, piroxicam, tenoxicam, ibuprofen, ketoprofen, naproxen, azapropazon, nabumetone, carprofen, tiaprofenic acid, suprofen, indoprofen, etodolac, fenoprofen, fenbufen, flurbiprofen, bermoprofen, pirazolac, zaltoprofen, nabumetone, bromfenac, ampiroxicam and lomoxicam. This list should however not be considered as exhaustive in any way.

The wording “NO-donating NSAID” is contemplated to include any non-steroidal anti-inflammatory drug (NSAID), a salt or an enantiomer thereof, which has the capability to release nitrogen oxide.

NO-donating NSAIDs that may be used in accordance with the present invention, are drugs of the formula I 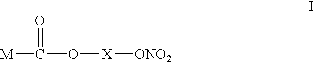
wherein:
X is a spacer, i.e. a drug forming a bridge between the nitrogen oxide donating group and the NSAID; and
M is selected from anyone of 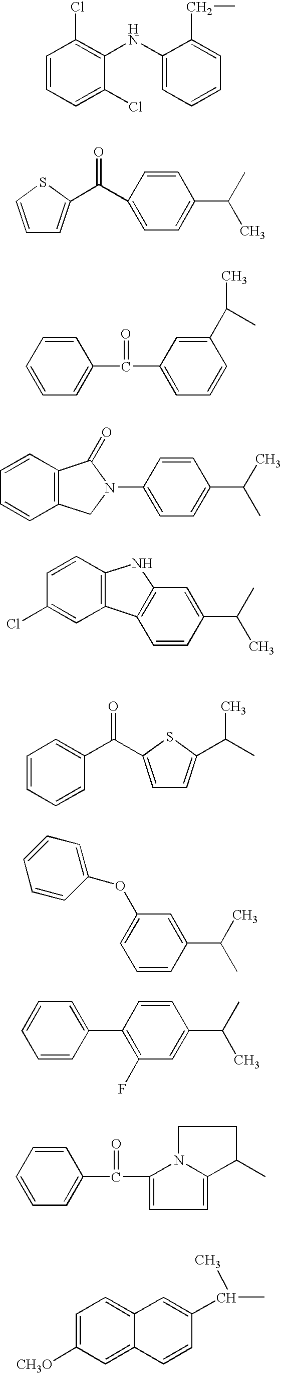 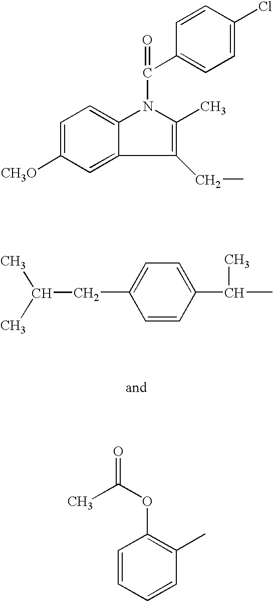
   or a salt or an enantiomer thereof.

In one embodiment of the invention, the spacer X is selected from a linear, branched or cyclic alkylene group —(CH₂)—ₙ wherein n is an integer from 2 to 10; and —(CH₂)ₘ—O—(CH₂)ₚ— wherein m and p are integers from 2 to 10; and —CH₂-pC₆H₄—CH₂— wherein p is an integer of from 2 to 10.

The spacer X may also contain a sulfur or heterocycle group.

The NO-donating NSAIDs contemplated as active drug(s) as well as processes for their preparation are disclosed in WO 94/04484, WO 94/12463, WO 95/09831 and WO 95/30641, which are hereby incorporated by reference. These documents also describe that the NO-donating NSA/Ds have an improved side-effect profile.

Further examples of active drug(s) that may be used in the composition of the present invention are compounds disclosed in WO 96/32946, WO 00/25776, EP 1126838, EP 821589, WO 02/60378, FR 2735366, FR 2737662, CN 1144092, WO 01/12584, WO 98/25918, WO 00/51988 and WO 00/06585, which are hereby incorporated by reference.

Specific NO-donating NSAIDs useful in accordance with the present invention are

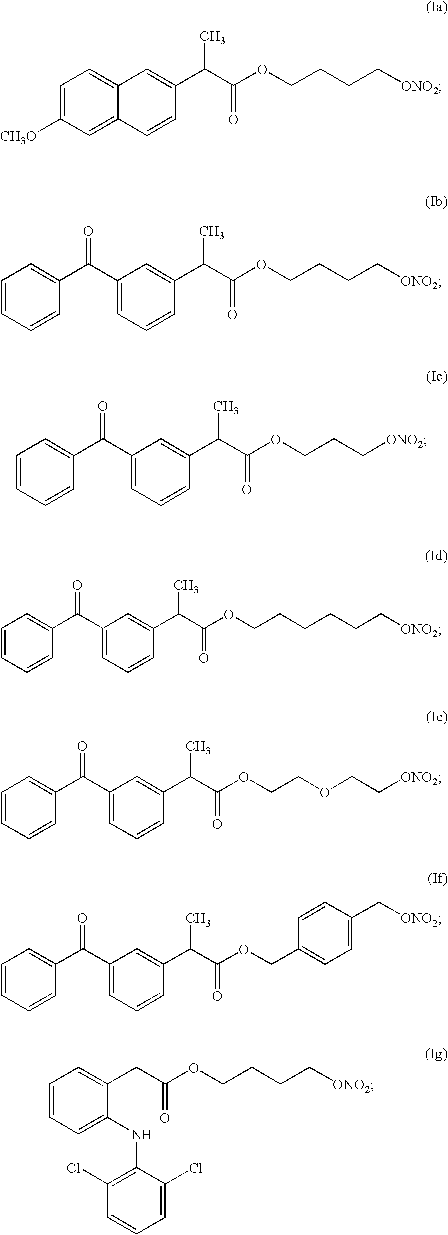

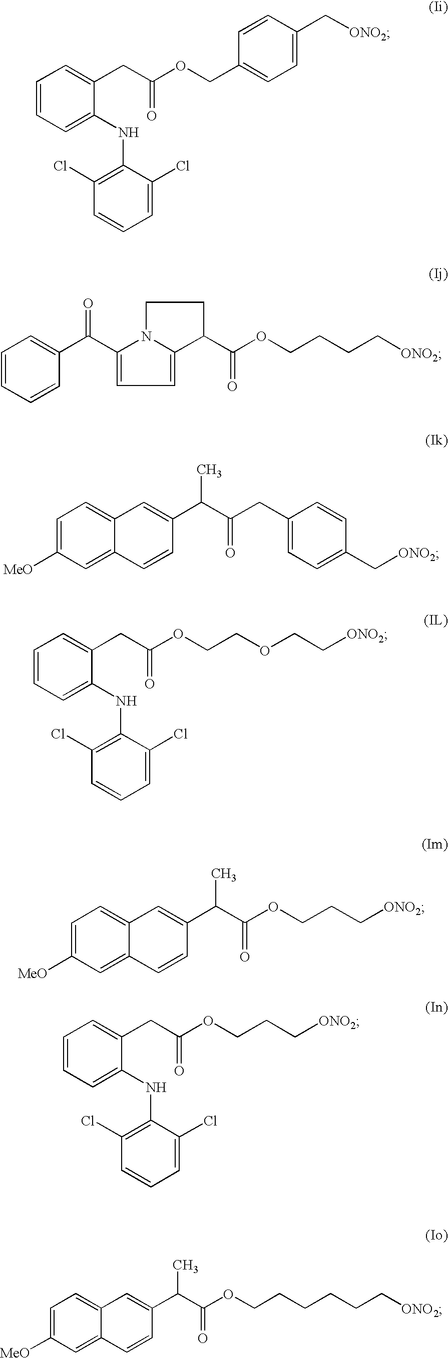

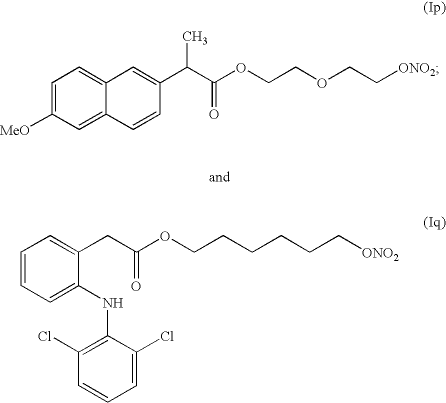

One embodiment of the invention relates to solid drug delivery composition comprising porous particles comprising one or more NO-donating NSA/D(s) wherein the NO-donating NSAID(s) is selected from an NO-donating naproxen, an NO-donating diclofenac and an NO-donating ketoprofen.

In another embodiment of the invention the NO-donating naproxen is 4-(nitrooxy)-butyl(S)-2-(9-methoxy-2-naphtyl)-propanoate (Compound of formula Ia).

In a further embodiment of the invention the NO-donating diclofenac is 2-[(2,6-dichlorophenyl)amino]benzeneacetic acid 4-(nitrooxy)-butyl ester (Compound of formula Ig).

In yet a further embodiment of the invention the NO-donating diclofenac is 2-[2-(nitrooxy)ethoxy]ethyl {2-[(2,6-dichlorophenyl)amino]phenyl}acetate (Compound of formula IL).

In another embodiment of the invention the NO-donating ketoprofen is 2-(3-benzoyl-phenyl)-propionic acid 3-nitrooxy-propyl ester or 2-(3-benzoyl-phenyl)-propionic acid 4-nitrooxymethyl-benzyl ester (Compound of formula Ic and Compound of formula If, respectively).

The Drug Delivery Composition

A new way of formulating the NO-donating NSAID(s) is to absorb it into porous carriers. Useful carriers for the NO-donating NSA/D(s) are carriers having properties such as a high oil-absorbing capacity, so that the drug easily absorbs into the carrier. Also, the carrier should have a satisfying liquid-holding ability, the volume of the active drug must be kept to guarantee the dose administered.

The invention relates to a solid drug delivery composition comprising one or more NO-donating Non Steroidal Antiinflammatory Compound(s) (NO-donating NSAID(s)) absorbed into porous particles.

The invention further relates to a solid drug delivery composition wherein one or more NO-donating NSAID(s) in oily form is absorbed into porous particles.

The invention also relates to a solid drug delivery composition wherein one or more NO-donating NSAID(s) in melted form is absorbed into porous particles.

The material of the porous particles used for absorbing the NO-donating NSAID(s) and for carrying the active drug in a drug delivery composition may be selected from materials such as, for example, calcium silicate, e.g. known under the trade name Florite™, dibasic calciumphosphate anhydrous, e.g.: known under trade name Fujicalin™, magnesium aluminometasilicate, e.g. known under trade name Neusilin™ and microcrystalline cellulose.

One embodiment of the invention relates to solid drug delivery composition according to the present invention wherein the porous particles are selected from the group consisting of dibasic calcium phosphate anhydrous, microcrystalline cellulose and pregelatinised starch or a mixture thereof.

The porous carriers exemplified above are free-flowing, which is advantageous during handling and preparation. The porous particle, comprising one or more NO-donating NSAID(s), may be used for direct compression into a multiple unit tableted delivery composition. Another example of a suitable dosage form is a capsule filled with the porous particles comprising one or more NO-donating NSAID(s). A further example of a suitable dosage form is a sachet comprising said the porous particles comprising one or more NO-donating NSAID(s).

The porous particle material used as carrier shall have a particle size between 50 and 500 μm, particularly a size between 100 and 150 μm.

Thus 95% of the particles used in the composition of the present invention shall have a size in the ranges mentioned above.

The liquid absorption capacity of the particles is suitably between 0.70 and 4.0 ml/g.

The pore size of the porous particles should be between 10 and 1000 Å, particularly between 20 and 750 Å, and most suitably between 50 and 500 Å.

Thus 95% of the particles used in the composition of the present invention shall have a pore size in the ranges mentioned above.

The invention relates to compositions wherein the NO-donating NSAID(s) have been absorbed into porous particles. Examples of suitable NO-donating NSAIDs are NO-donating naproxen, NO-donating diclofenac and NO-donating ketoprofen (according to Formula Ia, Ic, If, Ig and IL). The invention is not in any way restricted to compositions comprising these active drugs.

The NO-donating NSAIDs may be finely dispersed and absorbed into the porous particles either as the sole drug; as SEDDs; as a water-in-oil emulsion; as an oil-in-water emulsion; or as a dissolved or melted crystalline drug.

The releasing rate of the active drug from the composition may be influenced by the presence or absence of one or more surfactant(s). It has been shown that the release characteristics can be changed by adding one or more surfactant(s). The rate of release may be increased if a suitable surfactant is present together with the active drug into the porous particle.

The invention relates to a solid drug delivery composition comprising porous particles wherein one or more NO-donating NSAID(s) is absorbed together with one or more surfactant(s) into the porous particles.

Further, to control the release from the tablet, a SEDDS-mixture of the NO-donating drug may be absorbed into the porous particles.

Further, the present invention relates to a solid drug delivery composition comprising a combination of
a) porous particles comprising an NO-donating NSAID and one or more surfactant(s) and
b) porous particles comprising an NO-donating NSAID without surfactant.

Such a solid drug delivery composition will give a more advanced release profile, for example a first rapid onset by the release from the porous particles comprising the NO-donating NSAID with one or more surfactant(s) followed by a delayed release from the porous particles comprising the NO-donating NSA/D alone.

The NO-donating NSA/D(s) used for such a combination may be the same or different.

The wording “surfactant” is defined as surface-active amphiphilic drugs such as block co-polymers. suitable surfactants are non-ionic surfactants, for example those containing polyethylene glycol (PEG) chains, particularly block co-polymers such as poloxamers. One embodiment of the invention relates to polyoxyethylene polyoxybutylene block copolymer.

Examples of suitable poloxamers that may be used in the composition of the present invention are Poloxamer 407 (Pluronic F127™); Poloxamer 401 (Pluronic L121™); Poloxamer 237 (Pluronic F87™); Poloxamer 338 (Pluronic F108™); Poloxamer 331 (Pluronic L101™); Poloxamer 231 (Pluronic L81™); tetrafunctional polyoxyethylene polyoxypropylene block copolymer of ethylene diamine, e.g. known as Poloxamine 908 (Tetronic 908™); Poloxamine 1307 (Tetronic 1307™); Poloxamine 1107; polyoxyethylene polyoxybutylene block copolymer, e.g. known as Polyglycol BM45™. This list of poloxamers serves as examples of surfactants that may be used in the present invention, and should not in any way be considered as exhaustive or limiting the invention.

All surfactants described above are commercially available from, e.g. BASF, Dow Chemicals, and Gattefossé. The total amount of surfactant(s) may be within a range from 12.5 to 6000 mg, particularly from 100 to 500 mg.

The ratio NO-donating NSAID(s):surfactant(s) may vary from 1:0.1 to 1:10 (w/w), particularly from 1:0.3 to 1:3 (w/w).

Furhter, the solid drug delivery composition of the present invention may comprise a combination of one or more NO-donating NSA/D(s), optionally with one or more surfactant(s) and one or more other active drugs.

Preparation of the Drug Delivery Composition

The incorporation of the NO-donating NSAID(s), into the porous particles may be accomplished by conventional known methods.

Without Surfactant

The porous particles comprising one or more NO-donating NSAID(s) may be prepared in different ways, for example by mixing the NO-donating NSAID(s) with the porous particles directly, e.g. in a mortar.

Alternatively, the drug may be dissolved in a suitable solvent, such as one or more alcohol(s) for example, ethanol. The porous particles may than be added after which the active drug will be absorbed. The solvent(s) is then evaporated and the particles are collected. Furthermore, the NO-donating NSA/D(s) may be melted before mixing with the porous particles.

The invention relates to a process for producing the porous particles comprising one or more NO-donating NSAID(s) comprising mixing the NO-donating NSAID(s), optionally in oily or melted form, with porous particles.

One embodiment of the invention relates to a process for producing the porous particles comprising one or more NO-donating NSAID(s) comprising:
a) dissolving the NO-donating NSAID(s) in one or more alcohol(s),
b) adding the porous particles during stirring,
c) evaporating the added alcohol(s),
d) recovering the porous particles comprising the NO-donating NSAID(s), with a) and b) in optional order.

Another embodiment of the invention relates to a process for producing the porous particles comprising one or more NO-donating NSAID(s) comprising:
a) melting the NO-donating NSAID(s),
b) adding the porous particles,
c) stirring the obtained mixture,
d) recovering the porous particles comprising the NO-donating NSAID(s), with a) and b) in optional order.

The NO-donating NSAID(s) used in these processes may be the same or different.

With Sufactant

One or more surfactant(s) is added to the active drug before adding the porous particles.

The components may also be melted before mixing to get a homogeneous mixture of the two components before the addition of the porous particles.

The NO-donating NSAID(s) may be mixed with one or more liquid surfactant(s), and then absorbed into porous particles.

One embodiment of the present invention relates to a process for producing porous particles comprising one or more NO-donating NSAID(s) and one or more surfactant(s) comprising:
a) mixing NO-donating NSAID(s) and the surfactant(s),
b) adding the porous particles,
c) stirring the obtained mixture,
d) recovering the porous particles comprising NO-donating NSAID(s) and the surfactant(s),
   with a) and b) in optional order.

Another embodiment of the present invention relates to a process for producing the porous particles comprising one or more NO-donating NSAID(s) and the surfactant(s) comprising:
a) melting NO-donating NSAID(s) and the surfactant(s),
b) adding the porous particles,
c) stirring the obtained mixture,
d) recovering the porous particles comprising NO-donating NSAID(s) and the surfactant(s),
   with a) and b) in optional order.

The NO-donating NSAID(s) used in these processes may be the same or different.

Spheronization

The porous particles according to the invention may also be produced by way of spheronization. Speronization may be performed in any conventional way known to the man in the art. Pharmaceutical Pelletization Technology“ ”, Ed.: Isaac Ghebre-Sellasie, 1989, Marcel and Dekker, Inc.

One embodiment of the present invention relates to a process for producing the porous particles comprising one or more NO-donating NSAID(s) comprising:
a) mixing the NO-donating NSAID(s) and the porous excipient,
b) adding water, stepwise, continuously, in one portion,
c) extruding the obtained mixture into particles,
d) spheronising the obtained particles,
e) drying the obtained mixture,
f) recovering the porous particles comprising the NO-donating NSAID(s).

The NO-donating NSAID(s) in step a) may optionally be pre-heated.

The NO-donating NSAID(s) used in this processes may be the same or different.

The term ‘extruding’ shall mean forcing out, pushing out or expulsing.

The porous particles comprising the NO-donating NSAID(s) with or without surfactant(s), may be mixed with pharmaceutical acceptable excipients such as fillers, binders, disintegrants and/or pharmaceutically additives, carriers and diluents, before formulation in a suitable drug delivery composition.

The porous particles comprising the NO-donating NSAID(s) with or without surfactant(s), may also be used as such.

Optionally, the prepared porous particles comprising the NO-donating NSAID(s) with or without surfactant(s), may be mixed with a second active drug, for example, enteric coating layered pellets comprising a proton pump inhibitor.

Such a composition may be formulated by mixing the porous particles comprising the NO-donating NSAID(s) with or without surfactant(s), and the second active drug with pharmaceutical acceptable excipients such as fillers, binders, carriers, diluents, disintegrants and/or pharmaceutically additives followed by formulation of the obtained mixture into a suitable drug delivery composition.

Examples of suitable drug delivery composition are capsules and tablets. Tablets may be obtained by direct compressed.

Example of suitable excipients, but not limited thereto, are microcrystalline cellulose and polyvinyl pyrrolidone, hydroxypropyl methylcellulose (HPMC), lactose, sodium carboxymethylcelulolose (NaCMC).

The particles, capsules and tablets may be coated by ways well known in the art.

The filling into capsules, compressing to tablets and coating should preferably be performed in such a manner that does not substantially influence the release characteristics of the solid drug delivery composition after administration.

The prepared particles, capsules and tablets may be coated by a conventional film coat or a sugar coat, to obtain an improved appeareance. Suitable layering material for the film coat but not limited thereto, are derivatives of cellulose, such as hydroxypropylmethylcellulose, methylcellulose or ethylcellulose and acrylate-based polymers.

Sugar coating involves successive application of sucrose based solutions to the particles, capsules or tablets.

One embodiment of the present invention relates to a solid drug delivery composition wherein the porous particles are mixed with pharmaceutically acceptable excipients and compressed into a tablet.

A further embodiment of the present invention relates to a solid drug delivery composition wherein the porous particles are filled into a capsule.

Another embodiment of the present invention relates to a solid drug delivery composition wherein the capsules and tablets are coated.

If a fast release of the active drug is desired in the small intestine, the loaded porous particles may be enteric coated.

Some of the NO-donating NSAIDs may be high dose drugs. One embodiment of the invention relates to a divisable dosage form, for example, a divisable tablet.

The total amount of NO-donating NSAID(s) used in the composition of the invention is particularly in the range from 50 to 1500 mg per unit dose. The amount of a low dose NO-donating NSAID may be between 75 and 350 mg per unit dose. The amount of a high dose NO-donating NSA/D may be between between 350 and 1500 mg per unit dose.

The term “unit dose” is defined as the amount of active drug administered in one tablet, in one single capsule or a sachet.

Combinations

It is well known that patient compliance is an important factor in receiving an optimal result in medical treatments. An improved patient's compliance is obtained by administering different drugs in one dosage form.

One embodiment of the invention relates to a solid drug delivery composition comprising two or more different active drugs combined in one composition. Examples of a solid drug delivery composition in the form of a multiple unit tablet are shown. The solid drug delivery composition comprising such a combination may simplify the dosing regimen and improve patient compliance.

One embodiment of the invention relates to a mixture of porous particles comprising different NO-donating NSAIDs, with or without surfactant(s). For example, a combination of an NO-donating diclofenac and an NO-donating naproxen. This composition has an advanced release profile whereby the fast immediate release of NO-donating diclofenac is combined with the good maintenance of NO-donating naproxen.

Another embodiment of the invention relates to a combination of
a) porous particles comprising one or more NO-donating NSA/D(s), with or without surfactant(s) and
b) one or more other active drugs.

Each active drug may have a special requirement for being administered.

The NO-donating NSAID(s) may for example be combined with active drugs such as, anti-ulcer drugs. The porous particles comprising one or more NO-donating NSAID(s) according to the present invention may be combined with enteric coated pellets comprising a proton pump inhibitor, such as omeprazole. The H⁺, K⁺-ATPase inhibitors are one preferred group of drugs for combining with NO-donating NSAID(s). Examples of is specifically preferred drugs of H⁺, K⁺-ATPase inhibitors are acid susceptible proton pump inhibitors, for example drugs of the general formula II below. Although NO-donating NSAID(s) have an improved side-effect profile with respect to NSAID(s), the administration of NO-donating NSAID(s) together with a proton pump inhibitor may be a successful combination of drugs.

Compounds of formula II: 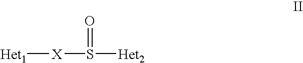
wherein:
Het₁ is 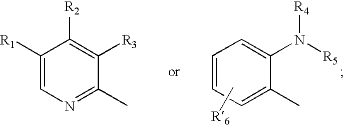
Het₂ is 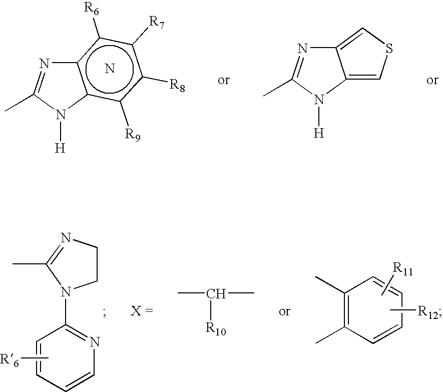
   and wherein
N in the benzimidazole moiety means that one of the carbon atoms substituted by R₆-R₉ optionally may be exchanged for a nitrogen atom without any substituents;
R₁, R₂ and R₃ are the same or different and selected from hydrogen, alkyl, alkoxy optionally substituted by fluorine, alkylthio, alkoxyalkoxy, dialkylamino, piperidino, morpholino, halogen, phenyl and phenylalkoxy;
R₄ and R₅ are the same or different and selected from hydrogen, alkyl and aralkyl;
R₆ is hydrogen, halogen, trifluoromethyl, alkyl and alkoxy;
R₆-R₉ are the same or different and selected from hydrogen, alkyl, alkoxy, halogen, haloalkoxy, alkylcarbonyl, alkoxycarbonyl, oxazolyl and trifluoroalkyl, or adjacent groups R₆-R₉ form ring structures, which may be further substituted;
R₁₀ is hydrogen or forms an alkylene chain together with R₃ and
R₁₁ and R₁₂ are the same or different and selected from hydrogen, halogen, alkyl and the alkyl groups.

The alkyl groups, alkoxy groups and moieties thereof, included in the substituents R₁-R₁₂ above may be branced or straight C₁-C₉-chains or comprise cyclic alkyl groups, such as cyclo-alkyl-alkyl.

Examples of proton pump inhibitors are omeprazole, esomeprazole, lansoprazole, pantoprazole or rabeprazole, leminoprazole or mixtures thereof. These examples are not in any way a restriction of possibilities.

The acid susceptible proton pump inhibitors used in the dosage forms of the invention may be used in their neutral form or in the form of an alkaline salt, such as for instance the Mg²⁺, Ca²⁺, Na⁺, K⁺ or Li⁺ salts, particularly the Mg²⁺ salts. Further, where applicable, the drugs listed above may be used in racemic form or in the form of the substantially pure enantiomer thereof, or alkaline salts of the single enantiomers.

Suitable proton pump inhibitors are for example disclosed in EP-A1-0005129, EP-A1-174 726, EP-A1-166 287, GB 2 163 747 and WO 90/06925, WO 91/19711, WO 91/19712, and further especially suitable drugs are described in WO 95/01977 (magnesium omeprazole) and WO 94/27988 (the single enantiomers of omeprazole salts).

The proton pump inhibitors used in a combination in accordance with the present invention are particularly provided as enteric coating layered pellets comprising the acid susceptible proton pump inhibitor. For the composition of the enteric coating layered pellets and its preparation, reference is made to WO 96/01623, which is hereby incorporated by reference.

One embodiment of the invention relates to a solid drug delivery composition wherein the porous particles comprising a NO-donating NSAID, optionally mixed with one or more surfactant(s), are mixed together with enteric coated pellets comprising a H⁺, K⁺-ATPase inhibitor.

Another embodiment of the invention relates to a solid drug delivery composition wherein the porous particles comprising an NO-donating naproxen, an NO-donating diclofenac, an NO-donating ketoprofen or an NO-donating ketorolac, optionally mixed with one or more surfactant(s), are mixed together with enteric coated pellets comprising omeprazole, esomeprazole, lansoprazole, pantoprazole or rabeprazole, leminoprazole or a pharmaceutical acceptable salt thereof.

Suitable combinations in accordance with the present invention are for instance an NO-donating NSAID of the formula Ia (NO-donating naproxen) and omeprazole or an alkaline salt of omeprazole, (S)-omeprazole; an NO-donating NSAID of the formula Ig (NO-donating-diclofenac) and omeprazole or an alkaline salt of omeprazole, (S)-omeprazole or an NO-donating NSAID of the formula IL (NO-donating diclofenac) and omeprazole or an alkaline salt of omeprazole, (S)-omeprazole.

Preparation of the Combination

One embodiment of the invention relates to a solid drug delivery composition comprising a proton pump inhibitor (in the form of a racemat, an alkaline salt or one of its single enantiomers) and one or more NO-donating NSAIDs characterized in that the individually enteric coating layered units containing the proton pump inhibitor and optionally containing alkaline reacting substances, are mixed with the porous particles comprising the absorbed NO-donating NSAID(s) prepared according to the present invention and pharmaceutically acceptable excipients. The NO-donating NSAID(s) and excipient may also be in the form of granules. The dry mixture of enteric coating layered units comprising the proton pump inhibitor and the porous particles comprising the NO-donating NSAID(s) are formulated into a suitable dosage delivery composition such as a tablet, capsule or a sachet.

With the expression “individual units” is meant small beads, porous particles, granules or pellets, in the following referred to as pellets of the acid susceptible proton pump inhibitor.

When the solid drug delivery composition is a tablet, care should be taken not to affect the acid resistance of the enteric coating layered pellets comprising the acid susceptible proton pump inhibitor significantly during the compaction process. In other words the mechanical properties, such as the flexibility and hardness as well as the thickness of the enteric coating layers(s), should be secured so that the requirements on enteric coated articles as specified in the United States Pharmacopeia are accomplished with, i.e. the acid resistances should not decrease more than 10% during the compression of the pellets into tablets.

The acid resistance is defined as the amount of proton pump inhibitor in tablets or pellets after being exposed to simulated gastric fluid USP, or the 0.1 M HCL (aq) relative to that of unexposed tablets and pellets, respectively. In test the individual tablets or pellets are exposed to simulated gastric fluid of a temperature of 37° C. The tablets disintegrate rapidly and release the enteric coating layered pellets to the medium. After two hours the enteric coating layered pellets are removed and analyzed for content of the proton pump inhibitor using High Performance Liquid Chromatography (HPLC).

Use

The invention relates to the use of the solid drug delivery composition for the manufacture of a medicament for treating pain.

The invention further relates to the use of the solid drug delivery composition according for the manufacture of a medicament for treating inflammation.

The invention relates further to a method for the treatment of pain comprising oral administration to a patient suffering therefrom a solid compound delivery composition according to the present invention.

The invention relates even further to a method for the treatment of inflammation comprising oral administration to a patient suffering therefrom a solid compound delivery composition according to the present invention.

### EXAMPLES

The invention will now be described in more detail by the following examples, which are not to be construed as limiting the invention in any way.

The examples show the processes for producing the solid drug delivery composition comprising porous particles comprising one or more NO-donating NSAID(s) and a solid drug delivery composition comprising porous particles comprising one or more NO-donating NSAID(s), optionally mixed with one or more surfactant(s). Also, an example showing a solid drug delivery composition of a combination of an NO-donating NSAID and the proton pump inhibitor omeprazole is presented.

The following porous materials were used in the examples: calcium silicate, dibasic calciumphosphate anhydrous (Fujicalin™) and magnesium aluminometasilicate (Neusilin™).

The following surfactants were used in the examples: Poloxamer 237 (Pluronic F87™) and Poloxamer 338 (Pluronic F108™).

The following microcrystalline cellulose was used in the examples: Avicel™ pH 102.

Characteristics of the Porous Particles (95%)

| | Pore size | Surface area | | Particle diameter | |
|---|---|---|---|---|---|
| Fujicalin ™ | 7.5 nm | 33 | m²/g | 40-150 | μm |
| Neusilin ™ | — | 110 | m²/g | 40-80 | μm |

Experiments of Compositions Comprising One NO-Donating NSAID

Free-flowing powders comprising porous particles comprising the active drug were made by mixing the active drug with porous particles as described below.

I. Compound of Formula Ia

The mixtures A to F were mixed with a pestle in a mortar at 60° C.

A) Compound of Formula Ia/Neusilin™ 1/1

12.50 g Compound of formula Ia
12.50 g Neusilin™
   B) Compound of Formula Ia/Fujicalin™ 1/2
8.33 g Compound of formula Ia
16.67 g Neusilin™
   C) Compound of Formula Ia/Neusilin™ 2/1
16.67 g Compound of formula Ia
8.33 g Neusilin™
   D) Compound of Formula Ia/Fujicalin™ 1/1.5
10 g Compound of formula Ia
15 g Fujicalin™
   E) Compound of Formula Ia/Fujicalin™ 1/1.25
11 g Compound of formula Ia
13.75 g Fujicalin™
   F) Compound of Formula Ia/Calcuim silikate 1/4
5 g Compound of formula Ia
20 g Calcuim silicate

The above mentioned mixtures A to F were sieved through a 0.5 mm sieve, and mixed with tablet excipients according to the excipient mixture 1 and 2 as described below. The compositions were compressed into tablets, weighing 1200 mg, using a tablet machine rigged with 18 mm oval punches.

Excipient Mixture 1

48.30 g Avicel™ pH 102
1.65 g Polyvinyl pyrrolidone, cross-linked
0.15 g Sodium stearyl fumarate
   Excipient Mixture 2
48.30 g Avicel™ pH 102
1.65 g Polyvinyl pyrrolidone, cross-linked
   Composition 1:
   Tablets, 640 mg Compound of Formula Ia
10 g Compound of formula Ia/Neusilin™ 2/1 (C)
2.5 g Excipient mixture 1.
   Composition 2:
   Tablets, 320 mg Compound of Formula Ia
10 g Compound of formula Ia/Fujicalin™ 1/2 (B)
2.5 g Excipient mixture 1
   Composition 3:
   Tablets, 200 mg Compound of Formula Ia
6 g Compound of formula Ia/Fujicalin 1/2 (B)
6 g Excipient mixture 1
   Composition 4:
   Tablets, 300 mg Compound of Formula Ia
6 g Compound of formula Ia/Neusilin™ 1/1 (A)
6 g Excipient mixture 1
   Composition 5:
   Tablets, 200 mg Compound of Formula Ia
6 g Compound of formula Ia/Neusilin™ 1/2 (B)
6 g Excipient mixture 1
   Composition 6:
   Tablets, 240 mg Compound of Formula Ia
6 g Compound of formula Ia/Fujicalin™ 1/1.5 (D)
6 g Excipient mixture 2
   Composition 7:
   Tablets, 267 mg Compound of Formula Ia
6 g Compound of formula Ia/Fujicalin™ 1/1.25 (E)
6 g Excipient mixture 2
   Composition 8:
   Tablets, 375 mg Compound of Formula Ia
9.38 g Compound of formula Ia/Fujicalin™ 1/1.5 (D)
2.62 g Excipient mixture 2
   Composition 9:
   Tablets, 375 mg Compound of Formula Ia
8.44 g Compound of formula Ia/Fujicalin™ 1/1.25 (E)
3.56 g Excipient mixture 2
   Composition 10:
   Tablets, 120 mg Compound of Formula Ia
10 g Compound of formula Ia/Calcuim silicate 1/4 (F)
10 g Excipient mixture 1
   Results—Compositions 1 to 10

The dissolution rate was determined by using a thermostated beaker with a magnetic stirrer (150 rpm). The dissolution medium had a temperature of 37° C. The media used was phosphate buffer pH=6.8, containing 8.8 mg/litre of CTAB. The increase in absorbance corresponded to the release of Compound of Formula Ia.

Composition 1. Tablet, 640 mg of Compound of Formula Ia

| | |
|---|---|
| Time | % Released |

| | | |
|---|---|---|
| 5 | min | 1 |
| 10 | min | 4.9 |
| 15 | min | 12.1 |
| 30 | min | 24.3 |
| 60 | min | 38.1 |

Composition 2. Tablet, 320 mg of Compound of Formula Ia

| | |
|---|---|
| Time | % Released |

| | | |
|---|---|---|
| 5 | min | 15 |
| 10 | min | 30 |
| 15 | min | 40 |
| 30 | min | 50 |
| 60 | min | 60 |

Composition 3. Tablet, 200 mg of Compound of Formula Ia

| | |
|---|---|
| Time | % Released |

| | | |
|---|---|---|
| 5 | min | 26.5 |
| 10 | min | 51.4 |
| 15 | min | 61.6 |
| 30 | min | 83 |
| 60 | min | 91 |

Composition 4. Tablet, 300 mg of Compound of Formula Ia

| | |
|---|---|
| Time | % Released |

| | | |
|---|---|---|
| 5 | min | 2 |
| 10 | min | 5.5 |
| 15 | min | 8.3 |
| 30 | min | 17.2 |
| 60 | min | 28.5 |

Composition 5. Tablet, 200 mg of Compound of Formula Ia,

| | |
|---|---|
| Time | % Released |

| | | |
|---|---|---|
| 5 | min | 1 |
| 10 | min | 1 |
| 15 | min | 2.3 |
| 30 | min | 5.7 |
| 60 | min | 9.1 |

Composition 6. Tablet, 240 mg of Compound of Formula Ia

| | |
|---|---|
| Time | % Released |

| | | |
|---|---|---|
| 5 | min | 31.5 |
| 10 | min | 51.9 |
| 15 | min | 63.1 |
| 30 | min | 83 |
| 60 | min | 98.2 |

Composition 7. Tablet, 267 mg of Compound of Formula Ia

| | |
|---|---|
| Time | % Released |

| | | |
|---|---|---|
| 5 | min | 26.7 |
| 10 | min | 43.6 |
| 15 | min | 56.5 |
| 30 | min | 78.9 |
| 60 | min | 97.8 |

Composition 8. Tablet, 375 mg of Compound of Formula Ia

| | |
|---|---|
| Time | % Released |

| | | |
|---|---|---|
| 5 | min | 19 |
| 10 | min | 30.5 |
| 15 | min | 37.5 |
| 30 | min | 52 |
| 60 | min | 59 |

Composition 9. Tablet, 375 mg of Compound of Formula Ia

| | |
|---|---|
| Time | % Released |

| | | |
|---|---|---|
| 5 | min | 18.9 |
| 10 | min | 31.5 |
| 15 | min | 40.5 |
| 30 | min | 51.6 |
| 60 | min | 62 |

Composition 10. Tablet, 120 mg of Compound of Formula Ia

| | |
|---|---|
| Time | % Released |

| | | |
|---|---|---|
| 10 | min | 23 |
| 20 | min | 37 |
| 30 | min | 47 |
| 40 | min | 55 |
| 60 | min | 67 |

Experiments of Compositions Comprising a Spheronised NO-Donating NSAID
G. Spheronised Compound of Formula Ia
200 g Compound of formula Ia
600 g Avicel™ pH 102
100+150+150+50 g water

The Avicel™ pH 102 was put in an intense mixer, the Compound of formula Ia was preheated to 45° C., and added to the Avicel™ pH 102 in the intensive mixer. After 3 minutes of mixing, water was added in portions stated above, under continuously mixing. Then the wet mass was extruded through a screen, diameter=1.0 mm. The extruded mass was then spheronised in a 0.325 mm spheroniser. The spheronised mass was then dried in a fluid bed at 45° C. for 5 minutes.

After the Compound of formula Ia had been spheronised and dried, an excipient mixture 2 was added.

Composition 11:

10 g Spheronised Compound of formula Ia (G)
10 g Excipient mixture 2

The tablet blend was sieved and then mixed in a Turbula for two minutes.

The obtained mixture was compressed into tablets, weighing 800 mg (corresponding to 92 mg of active), using a tablet machine rigged with 18 mm oval punches.

Result—Composition 11

Dissolution tests were made using a thermostated beaker with a magnetic stirrer (300 rpm).

The media used was phosphate buffer pH=6.8, containing 8.8 mg/litre of CTAB. The increase in absorbance corresponded to the release of Compound of formula Ia.

Composition 11. Tablet, 92 mg of Compound of Formula Ia

| | |
|---|---|
| Time | % Released |

| | | |
|---|---|---|
| 5 | min | 5 |
| 10 | min | 23 |
| 15 | min | 30 |
| 30 | min | 46 |
| 60 | min | 67 |

Experiments of Compositions Comprising One NO-Donating NSAID with One or More Surfactant(s)
1. Compound of Formula Ia

A mixture of Compound of formula Ia and one or more surfactant(s) was prepared by melting and mixing the surfactant(s) and the active drug at 60° C.

A free-flowing powder comprising Compound of formula Ia was made by adding the mixture to porous particles and mixing the components with a pestle in a mortar at 60° C.

H. Compound of Formula Ia/Pluronic F87™ 1/0.3

4 g Compound of formula Ia
1.2 g Pluronic F87™
   I. (Compound of Formula Ia/Pluronic F87™ 1/0.3)/Fujicalin™ 1/4
2 g Compound of formula Ia/Pluronic F87™ (H)
8 g Fujicalin™

The above mentioned mixtures H and I were sieved through a 0.5 mm sieve and mixed with excipients according to excipient mixture 2 as described below.

The compositions were compressed into tablets, weighing 1200 mg, using a tablet machine rigged with 18 mm oval punches.

Composition 12:

Tablets, 92 mg Compound of Formula Ia

5 g (Compound of formula Ia/Pluronic F87™ 1/0.3)/Fujicalin™ 1/4 (I)
5 g Excipient mixture 2
   Results—Composition 12

The dissolution rate was detemined in a thermostated beaker with a magentic stirrer (150 rpm). The media used was phosphate buffer pH-=6.8, containing 8.8 mg/litre of CTAB. The increase in absorbance corresponded to the release of Compound of formula Ia.

Composition 12. Tablet, 92 mg of Compound of formula Ia

| | |
|---|---|
| Time | % Released |

| | | |
|---|---|---|
| 5 | min | 80 |
| 10 | min | 96 |
| 15 | min | 99 |
| 30 | min | 100 |
| 60 | min | 100 |

2. Compound of Formula IL

Compound of formula IL was melted and thereafter mixed with the porous particles. After the hot melt had been absorbed into the porous particles, the excipient mixture 2 was added.

J. Compound of Formula IL/Fujicalin™ 1/2

2.5 g Compound of formula IL
5 g Fujicalin™
   Composition 13:
   Tablets, 100 mg of Compound of Formula IL
6 g Compound of formula IL Fujicalin™ (J)
6 g Excipient mixture 2

The tablet blend was sieved and then mixed in a Turbula for two minutes.

The obtained mixture was compressed into tablets, weighing 600 mg (corresponding to 100 mg of Compound of formula IL), using a tablet machine rigged with 18 mm oval punches.

Result—Composition 13

Dissolution tests were made using a USP paddle bath (USP dissolution test No. 2) operating at 50 rpm. The media used was phosphate buffer pH=6.8, containing 8.8 mg/litre of CTAB. The increase in absorbance corresponded to the release of Compound of formula IL.

Composition 13. Tablet, 100 mg of Compound of formula IL

| | |
|---|---|
| Time | % Released |

| | | |
|---|---|---|
| 5 | min | 21 |
| 10 | min | 33 |
| 15 | min | 40 |
| 30 | min | 56 |
| 60 | min | 70 |

K. (Compound of Formula IL/Pluronic F108™ 1/0.3)/Fujicalin™ 1/2
3.0 g Compound of formula IL
0.90 g Pluronic F108™
7.80 g Fujicalin™

The active drug and the surfactants were melted together and thereafter mixed with the porous particles. After the hot melt (containing both the active drug and surfactants) had been absorbed into the porous particles, the excipient mixture 2 was added.

Composition 14:

Tablets, 100 mg of Compound of Formula IL

7.80 g (Compound of formula IL Pluronic F108™ 1/0.3)/Fujicalin™ 1/2 (K)
7.80 g Excipient mixture 2

The tablet blend was sieved and then mixed in a Turbula for two minutes.

The obtained mixture was compressed into tablets, weighing 780 mg (corresponding to 100 mg of Compound of formula IL), using a tablet machine rigged with 18 mm oval punches.

Result—Composition 14

Dissolution tests were made using a thermostated beaker with a magnetic stirrer (300 rpm).

The media used was phosphate buffer pH=6.8, containing 8.8 mg/litre of CTAB. The increase in absorbance corresponded to the release of Compound of formula IL.

Composition 14. Tablet, 100 mg of Compound of formula IL

| | |
|---|---|
| Time | % Released |

| | | |
|---|---|---|
| 5 | min | 18 |
| 10 | min | 31 |
| 15 | min | 40 |
| 30 | min | 73 |
| 60 | min | 100 |

3. Compound of Formula Ic
L. Compound of Formula Ic/Fujicalin™ 1/2
2.5 g Compound of formula Ic
5 g Fujicalin™

The Compound of formula Ic was mixed with the porous particles. After Compound of formula Ic had been absorbed into the porous particles, the excipients mixture 2 was added.

Composition 15:

Tablets, 100 mg of Compound of Formula Ic

6 g Compound of formula Ic/Fujicalin™ (L)
6 g Excipient mixture 2

The tablet blend was sieved and then mixed in a Turbula for two minutes.

The obtained mixture was compressed into tablets, weighing 600 mg (corresponding to 100 mg of Compound of formula Ic), using a tablet machine rigged with 18 mm oval punches.

Result—Composition 15

Dissolution tests were made using a thermostated beaker with a magnetic stirrer (300 rpm). The media used was phosphate buffer pH=6.8, containing 8.8 mg/litre of CTAB. The increase in absorbance corresponded to the release of Compound of formula Ic.

Composition 15. Tablet, 100 mg of Compound of formula Ic

| | |
|---|---|
| Time | % Released |

| | | |
|---|---|---|
| 5 | min | 13 |
| 10 | min | 22 |
| 15 | min | 29 |
| 30 | min | 35 |
| 60 | min | 39 |

M. (Compound of Formula Ic/Pluronic F108™ 1/0.3)/Fujicalin™ 1/2
3.0 g Compound of formula Ic
0.90 g Pluronic F108™
7.80 g Fujicalin™

The active drug and surfactants were melted and mixed together and thereafter added to the porous particles. After the hot melt (containing both drug and surfactants) had been absorbed into the porous particles, the excipients mixture 2 was added.

Composition 16:

Tablets, 100 mg of Compound of Formula Ic

7.80 g (Compound of formula Ic/Pluronic F108™ 1/0.3)/Fujicalin™ (M)
7.80 g Excipient mixture 2

The tablet blend was sieved and then mixed in a Turbula for two minutes.

The obtained mixture was compressed into tablets, weighing 780 mg (corresponding to 100 mg of Compound of formula Ic), using a tablet machine rigged with 18 mm oval punches.

Result—Composition 16

Dissolution tests were made using a thermostated beaker with a magnetic stirrer (300 rpm). The media used was phosphate buffer pH=6.8, containing 8.8 mg/litre of CTAB. The increase in absorbance corresponded to the release of Compound of formula Ic.

Composition 16. Tablet, 100 mg of Compound of formula Ic

| | |
|---|---|
| Time | % Released |

| | | |
|---|---|---|
| 5 | min | 21 |
| 10 | min | 24 |
| 15 | min | 29 |
| 30 | min | 36 |
| 60 | min | 39 |

4. Compound of Formula If
N. Compound of Formula If/Fujicalin™ 1/2
2.5 g Compound of formula If
5 g Fujicalin™

The active drug (an oil) was mixed with the porous particles. After the active had been absorbed into the porous particles, the excipients mixture 2 was added.

Composition 17:

Tablets, 100 mg of Compound of Formula If

6 g Compound of formula If/Fujicalin™ (N)
6 g Excipient mixture 2

The tablet blend was sieved and then mixed in a Turbula for two minutes.

The obtained mixture was compressed into tablets, weighing 600 mg (corresponding to 100 mg of Compound of formula If), using a tablet machine rigged with 18 mm oval punches.

Result—Composition 17

Dissolution tests were made using a thermostated beaker with a magnetic stirrer (300 rpm). The media used was phosphate buffer pH=6.8, containing 8.8 mg/litre of CTAB. The increase in absorbance corresponded to the release of Compound of formula If.

Composition 17. Tablet, 100 mg of Compound of formula If

| | |
|---|---|
| Time | % Released |

| | | |
|---|---|---|
| 5 | min | 15 |
| 10 | min | 22 |
| 15 | min | 25 |
| 30 | min | 30 |
| 60 | min | 40 |

O. (Compound of Formula If/Pluronic F108™ 1/0.3)/Fujicalin™ 1/2
3.0 g Compound of formula If
0.90 g Pluronic F108™
7.80 g Fujicalin™

The Compound of formula If and surfactant were melted together and thereafter mixed with the porous particles. After the hot melt (containing both drug and surfactant) had been absorbed into the porous particles, the excipient mixture 2 was added.

Composition 18:

Tablets, 100 mg of Compound of Formula If

7.80 g (Compound of formula If/Pluronic F108™ 1/0.3)/Fujicalin™ (O)
7.80 g Excipient mixture 2

The tablet blend was sieved and then mixed in a Turbula for two minutes.

The obtained mixture was compressed into tablets, weighing 780 mg (corresponding to 100 mg of Compound of formula If), using a tablet machine rigged with 18 mm oval punches.

Result—Composition 18

Dissolution tests were made using a thermostated beaker with a magnetic stirrer (300 rpm).

The media used was phosphate buffer pH=6.8, containing 8.8 mg/litre of CTAB. The increase in absorbance corresponded to the release of Compound of formula If.

Composition 18. Tablet, 100 mg of Compound of formula If

| | |
|---|---|
| Time | % Released |

| | | |
|---|---|---|
| 5 | min | 18 |
| 10 | min | 44 |
| 15 | min | 59 |
| 30 | min | 83 |
| 60 | min | 100 |

Experiments of Compositions Comprising More than One NO-Donating NSAID

Experiments were performed with compositions comprising Compound of Formula Ig and Compound of Formula Ia, mixed with one or more surfactant(s). Free-flowing powder comprising Compound of formula Ig was made by mixing the below mentioned composition P.

P. Compound of Formula Ig/Fujicalin™ 1/2

3 g Compound of formula Ig
6 g Fujicalin™

A mixture of Compound of formula Ia and a surfactant was prepared by melting and mixing the surfactant and the active drug with a pestle in a mortar at 60° C.

Q. Compound of Formula Ia/Pluronic F87™ 1/0.3

3.08 g Compound of formula Ia
0.92 g Pluronic F87™

Free-flowing powder comprising Compound of formula Ia was made by adding the above mentioned mixture (Q) to porous particles, using a pestle in a mortar at 60° C.

R. (Compound of Formula Ia/Pluronic F87™ 1/0.3)/Fujicalin 1/3

3 g Compound of formula Ia/Pluronic F87™ (Q)
9 g Fujicalin™

The above mentioned mixtures were sieved through a 0.5 mm sieve and mixed with excipient mixture 2.

The compositions were compressed into tablets, weighing 1200 mg, using a tablet machine rigged with 18 mm oval punches.

Composition 19:

Tablets, 120 mg of Compound of Formula Ig

3.60 g Compound of formula Ig/Fujicalin™ 1/2 (P)
8.40 g Excipient mixture 2
   Composition 20:
   Tablets, 120 mg of Compound of Formula Ia
6.24 g Compound of formula Ia/Pluronic F87™ (Q)
5.76 g Excipient mixture 2
   Composition 21:
   Tablets, 120 mg of Compound of formula Ig and Compound of Formula Ia
1.80 g Compound of formula Ig/Fujicalin™ (P)
3.12 g Compound of formula Ia/Pluronic F87™ (Q)
7.08 g Excipient mixture 2
   Results—Composition 19 to 21

Dissolution tests were made using a thermostated beaker with a stirrer (150 rpm). The media used was phosphate buffer pH=6.8, containing 8.8 mg/litre of CTAB. The increase in absorbance corresponded to the release of Compound of formula Ig and Compound of formula Ia.

Composition 19: Tablet, 120 mg of Compound of formula Ig

| | |
|---|---|
| Time | % Released |

| | | |
|---|---|---|
| 5 | min | 20 |
| 10 | min | 37 |
| 15 | min | 44 |
| 30 | min | 78 |
| 60 | min | 100 |

Composition 20: Tablet, 120 mg of Compound of formula Ia

| | |
|---|---|
| Time | % Released |

| | | |
|---|---|---|
| 5 | min | 60 |
| 10 | min | 77 |
| 15 | min | 82 |
| 30 | min | 100 |
| 60 | min | 100 |

Composition 21: Tablet, 120 mg of Compound
of formula Ig and Compound of formula Ia

| | |
|---|---|
| Time | % Released |

| | | |
|---|---|---|
| 5 | min | 60 |
| 10 | min | 80 |
| 15 | min | 100 |
| 30 | min | 100 |
| 60 | min | 100 |

Experiments of Compositions Comprising a Combination of a NO-Donating NSAID and a H⁺, K⁺-ATPase Inhibitor.

The following experiment show a composition comprising the Compound of formula Ia absorbed into a porous particle and mixed with enteric coated pellets comprising an acid susceptible proton pump inhibitor.

Composition 22:

A solid drug delivery composition in the form of a tablet comprising 250 mg of Compound of formula Ia and omeprazole 20 mg (as Mg-Omeprazole).

Enteric overcoated pellets comprising omeprazole and a powder of the porous particles comprising the Compound of formula Ia were manufactured separately, before compressing the two components together with pharmaceutically acceptable excipients into tablets.

Free-flowing powder of porous particles comprising Compound of formula Ia was made by making a mixture of

| | | |
|---|---|---|
| Compound of formula Ia | 250 | parts by weight |
| Calcium silicate | 250 | parts by weight |

in a mortar and working these drugs together. This mixture (500 parts by weight), a free-flowing powder, was sieved through a 0.5 mm sieve.

The enteric coated omeprazole pellet was made using the following ingredients:

| Core material (omeprazole) | | |
|---|---|---|
| Magnesium omeprazole | 15.00 | kg |
| Non-pareil seeds | 15.00 | kg |
| Hydroxypropyl methylcellulose | 2.25 | kg |
| Water purified | 40 | kg |

| Application of separating layer | | |
|---|---|---|
| Core material (acc. to above) | 15.00 | kg |
| Hydroxypropyl cellulose | 1.50 | kg |
| Talc | 2.57 | kg |
| Magnesium Stearate | 0.21 | kg |
| Water purified | 20 | kg |

| Enteric coating(omeprazole) | | |
|---|---|---|
| Separating layered pellets (acc. to above) | 18.00 | kg |
| Methacrylic acid copolymer (30% suspension) | 7.92 | kg |
| Triethyl citrate | 2.38 | kg |
| Mono- and diglycerides (NF) | 0.40 | kg |
| Polysorbate 80 | 0.04 | kg |
| Water purified | 17 | kg |

| Over-coating(omeprazole) | | |
|---|---|---|
| Enteric coated pellets | 25.00 | kg |
| Hydroxypropyl methylcellulose | 0.31 | kg |
| Mg-Stearate | 0.009 | kg |
| Water purified | 6 | kg |

The suspension layering was performed in a fluid bed apparatus. Magnesium omeprazole was sprayed onto inert non-pareil seeds from a water suspension containing the dissolved binder, i.e. hydroxypropylmethylcellulose.

The prepared core material was provided with a separating layer in a fluid bed apparatus with a hydroxypropyl cellulose solution containing talc and magnesium stearate. The enteric coating consisting of methacrylic acid copolymer, mono- and diglycerides, triethylcitrate and polysorbate was sprayed onto the separating layered pellets in a fluid bed apparatus. In the same type of apparatus the enteric coated pellets were coated with a hydroxypropyl methylcellulose/Mg-Stearate suspension. The over-coated pellets were sieved to remove possible agglomerates. Average medium particle size of the obtained pellets was around 0.5 mm in diameter.

The free-flowing powder comprising the porous particles comprising Compound of formula Ia was (500 parts by weight) mixed with:

| | | |
|---|---|---|
| Enteric overcoated omeprazole pellets (from above) | 100 | parts by weight |
| Microcrystalline cellulose (Avicel pH 102 special coarse grade) | 482 | parts by weight |
| Polyvinyl pyrrolidone, cross-linked | 16.5 | parts by weight |
| Sodium stearylfumarate | 1.5 | parts by weight |

Enteric overcoated omeprazole pellets were manufactured by charging components in proportions according to the recipe below;

The obtained mixture of porous particles comprising the absorbed Compound of formula Ia and the enteric coated pellets comprising omeprazole was compressed in a tabletting machine to tablets having an average weight of 1095 mg, using 20 mm in diameter flat punches. Tablet hardness was 5-6 kP.

Results—Composition 22

Omeprazole release was tested in USP dissolution apparatus No. 2 (paddle), operated at 100 rpm. After preexposure to simulated gastric juice for 2 hours the release was measured in 900 ml of phosphate buffer having a pH of 6.8, and after 30 minutes 90% of stated amount had been released.

For the release of drug of formula Ia, the same kind of apparatus and method as above was used and operated at 100 rpm. As dissolution media 1000 ml of phospahte buffer having a pH of 6.8 and also containing 8.8 mg/ml of CTAB was used.

The release was followed spectrophotometrically at 269 nm. The absorbance increase corresponded to the following release of Compound of formula Ia.

Composition 22, Tablet 250 mg of Compound of formula Ia

| | |
|---|---|
| Time | % Released |

| | | |
|---|---|---|
| 30 | min | 36 |
| 60 | min | 77 |
| 90 | min | 86 |
| 120 | min | 92 |
| 180 | min | 99 |

List of Abbreviations
USP United States Pharmacopeia
CTAB cetyltrimethylammonium bromide

1. A solid drug delivery composition comprising one or more NO-donating Non Steroidal Antiinflammatory Compound (s) (NO-donating NSAID(s)) absorbed into porous particles.

2. The solid drug delivery composition according to claim 1 wherein one or more NO-donating NSAID(s) in oily form is absorbed into porous particles.

3. The solid drug delivery composition according to claim 1 wherein one or more NO-donating NSAID(s) in melted form is absorbed into porous particles.

4. The solid drug delivery composition according to claim 1 wherein the porous particles are selected from the group consisting of dibasic calcium phosphate, anhydrous, microcrystalline cellulose and pregelatinised starch or a mixture thereof.

5. The solid drug delivery composition according to claim 1 wherein the porous particles are spherical with a particle size between 50 and 500 um.

6. The solid drug delivery composition according to claim 5 wherein the particle size of the spherical porous particles is between 100 and 150 um.

7. The solid drug delivery composition according to claim 1 wherein the pore size of the porous particles is between 10 and 1000 Å.

8. The solid drug delivery composition according to claim 7 wherein the pore size of the porous particles is between 20 and 750 Å.

9. The solid drug delivery composition according to claim 8 wherein the pore size of the porous particles is between 50 and 500 Å.

10. The solid drug delivery composition according to claim 1 wherein one or more NO-donating NSAID(s) is absorbed together with one or more surfactant (s) into the porous particles.

11. The solid drug delivery composition according to claim 1 comprising a combinations of
a) porous particles comprising an NO-donating NSAID and one or more surfactant(s) and
b) porous particles comprising an NO-donating NSAID without surfactant.

12. The solid drug delivery composition according to claim 10 wherein the NO-donating NSAID(s) are the same.

13. The solid drug delivery composition according to claim 10 wherein the surfactant (s) is non-ionic.

14. The solid drug delivery composition according to claim 13 wherein the surfactant(s) is a block co-polymer.

15. The solid drug delivery composition according to claim 13 wherein the surfactant(s) is a poloxamer.

16. The solid drug delivery composition according to claim 13 wherein the surfactant(s) is a polyoxyethylene polyoxybutylene block copolymer.

17. The solid drug delivery composition according to claim 10 wherein the ratio NO-donating NSAID(s): surfactant(s) is within the range from 1:0.1 to 1:10 (w/w).

18. The solid drug delivery composition according to claim 17 wherein the ratio NO-donating NSAID(s): surfactant(s) is within the range from 1:0.3 to 1:3 (w/w).

19. The solid drug delivery composition according to claim 1 wherein the NO-donating NSAID is an NO-donating naproxen.

20. The solid drug delivery composition according to claim 19 wherein the NO-donating naproxen is 4-(nitrooxy)butyl-(S)-2-(9-methoxy-2-naphtyl)-propanoate.

21. The solid drug delivery composition according to claim 1 wherein the NO-donating NSAID is an NO-donating diclofenac.

22. The solid drug delivery composition according to claim 21 wherein the NO-donating diclofenac is 2-[(2,6-dichlorophenyl)amino]benzeneacetic acid 4-(nitrooxy)-butyl ester.

23. The solid drug delivery composition according to claim 21 wherein the NO-donating diclofenac is 2-[2-(nitrooxy)ethoxy]ethyl {2-[(2,6-dichlorophenyl)amino]phenyl}acetate.

24. The solid drug delivery-composition according to claim 1 wherein the NO-donating NSAID is an NO-donating ketoprofen.

25. The solid drug delivery composition according to claim 24 wherein the NO-donating ketoprofen is 2-(3-benzoyl-phenyl)-propionic acid 3-nitrooxy-propyl ester or 2-(3-benzoyl-phenyl)-propionic acid 4-nitrooxymethyl-benzyl ester.

26. The solid drug delivery composition according to claim 1 wherein the porous particles comprising an NO-donating NSAID, optionally mixed with one or more surfactant(s), are mixed together with enteric coated pellets comprising a H+, K+-ATPase inhibitor.

27. The solid drug delivery composition according to claim 26 wherein the porous particles comprising an NO-donating naproxen, an NO-donating diclofenac, an NO-donating ketoprofen or an NO-donating ketorolac, optionally mixed with one or more surfactant(s), are mixed together with enteric coated pellets comprising omeprazole, esomeprazole, lansoprazole, pantoprazole or rabeprazole, leminoprazole or a pharmaceutical acceptable salt thereof.

28. Process for producing the porous particles comprising one or more NO-donating NSAID(s) according to claim 1 comprising mixing the NO-donating NSAID(s), optionally in oily or melted form, with porous particles.

29. Process for producing the porous particles comprising one or more NO-donating NSAID(s) according to claim 1 comprising:
a) dissolving the NO-donating NSAID(s) in one or more alcohol(s),
b) adding the porous particles during stirring,
c) evaporating the added alcohol(s),
d) recovering the porous particles comprising the NO-donating NSAID(s),
with a) and b) in optional order.

30. Process for producing the porous particles comprising one or more NO-donating NSAID(s) according to claim 1 comprising:
a) melting the NO-donating NSAID(s),
b) adding the porous particles,
c) stirring the obtained mixture,
d) recovering the porous particles comprising the NO-donating NSAID(s),
with a) and b) in optional order.

31. Process for producing porous particles comprising one or more NO-donating NSAID(s) and one or more surfactant(s) according to claim 1 comprising:
a) mixing the NO-donating NSAID(s) and the surfactant(s),
b) adding the porous particles,
c) stirring the obtained mixture,
d) recovering the porous particles comprising the NO-donating NSAID(s) and the surfactant(s),
with a) and b) in optional order.

32. Process for producing the porous particles comprising one or more NO-donating NSAID(s) and one or more surfactant(s) according to claim 1 comprising:
a) melting NO-donating NSAID(s) and the surfactant(s),
b) adding the porous particles,
c) stirring the obtained mixture,
d) recovering the porous particles comprising NO-donating NSAID(s) and the surfactant(s),
with a) and b) in optional order.

33. Process for producing the porous particles comprising one or more NO-donating NSAID(s) according to claim 1 comprising:
a) mixing the NO-donating NSAID(s) and the porous excipient,
b) adding water, stepwise, continuously, in one portion,
c) extruding the obtained mixture into particles,
d) spheronising the obtained particles,
e) drying the obtained mixture,
f) recovering the porous particles comprising the NO-donating NSAID(s).

34. The process according to claim 33 wherein the NO-donating NSAID(s) in step a) is pre-heated.

35. The process according to claim 28 wherein the NO-donating NSAID(s) are the same.

36. A solid drug delivery composition comprising one or more NO-donating Non Steroidal Antiinflammatory Compound(s) (NO-donating NSAID(s)) absorbed into porous particles wherein the porous particles have been produced according to claim 28, are mixed with pharmaceutically acceptable excipients and compressed into a tablet.

37. A solid drug delivery composition comprising one or more NO-donating Non Steroidal Antiinflammatory Compound(s) (NO-donating NSAID(s)) absorbed into porous particles wherein the porous particles have been produced according to claim 28, are filled into a capsule.

38. The solid drug delivery composition according to claim 36 wherein the capsules or tablets are coated.

39. Use of the solid drug delivery composition according to claim 1 for the manufacture of a medicament for treating pain.

40. Use of the solid drug delivery composition according to claim 1 for the manufacture of a medicament for treating inflammation.

41. A method for the treatment of pain comprising oral administration to a patient suffering therefrom a solid compound delivery composition according to claim 1.

42. A method for the treatment of inflammation comprising oral administration to a patient suffering therefrom a solid compound delivery composition according to claim 1.
